(19) United States
(12) Patent Application Publication
(10) Pub. No.: US 2026/0325709 A1
(43) Pub. Date: Oct. 22, 2026

(54) Engineered biodegradable vascular bioprostheses and production process thereof

(71) Applicant: Università degli Studi di Genova, Genova (IT)

(72) Inventors: Patrizia Perego, Genova (IT); Domenico Palombo, Genova (IT); Pier Francesco Ferrari, Genova (IT); Bahar Aliakbarian, Genova (IT); Bianca Pane, Genova (IT); Giovanni Salvatore Giuseppe Spinella, Genova (IT)

(21) Appl. No.: 19/308,193

(22) Filed: Aug. 23, 2025

Related U.S. Application Data
(63) Continuation-in-part of application No. 17/627,960, filed on Aug. 20, 2020, now abandoned.

(30) Foreign Application Priority Data
Aug. 23, 2019 (IT) 102019000014985

(51) Int. Cl. A61L 27/18; A61L 27/22; A61L 27/24; A61L 27/54; A61L 27/58; C08L 67/02; C08L 67/04

(52) U.S. Cl. CPC A61L 27/18; A61L 27/222; A61L 27/227; A61L 27/24; A61L 27/54; A61L 27/58; C08L 67/025; C08L 67/04; A61L 2300/41; A61L 2300/45; A61L 2300/604; A61L 2430/20; A61L 2430/22

(57) ABSTRACT

Engineered biodegradable vascular bioprostheses are made from a polymeric construct containing a mixture of at least two polymers, polycaprolactone (PCL) and poly(glycerol sebacate) (PGS), that include antioxidant bioactive molecules (biomolecules) causing a modulation of inflammation and proteins providing cell recruiting and trans-differentiation activities. The process for obtaining such engineered biodegradable vascular bioprostheses includes the preparation of a polymer solution by solubilizing at least two polymers in mixtures of organic solvents, electrospinning the polymer solution, and bioengineering the prostheses.

### FIELD OF THE INVENTION

The present invention relates to engineered biodegradable vascular bioprostheses and their production process. More particularly, the present invention relates to engineered biodegradable vascular bioprostheses made from a construct containing a mixture of at least two polymers, polycaprolactone (PCL) and poly(glycerol sebacate) (PGS), and having bioactive molecules (biomolecules) selected among antioxidants that modulate inflammation and among proteins that provide cell recruiting and trans-differentiation activities.

### BACKGROUND OF THE INVENTION

The implant of commercially available prostheses in patients suffering from peripheral obstructive vascular diseases generally generates an inflammatory-scarring process in most cases, which leads to the transformation of the prosthetic implant into a rigid and non-endothelialized structure, thus promoting thrombotic processes, especially for small caliber prostheses, and, in the case of germ implantation, during bacteremia or septicemia, the inability to eradicate the infection without explantation. To reduce and modulate the inflammatory and thrombotic responses, targeted therapeutic protocols are employed. Furthermore, these commercial prostheses are not biodegradable, not bioactive, and are not ideally suitable for the replacement of small caliber blood vessels. For these reasons, the use of biodegradable and engineered prostheses (bioprostheses) could ensure greater durability and patency of the implant over time. The success of the vascular bioprosthesis implant is also strictly dependent on its biodegradability, i.e., the time interval during which it will perform its function before being replaced by a neo-vessel, without undergoing thrombosis and calcification processes.

For these reasons, various methods have been defined to date, with limited success, for the production of small caliber biodegradable vascular prostheses. Among the synthesis techniques, the limited literature on the subject mentions electrospinning with all its variants, 3D printing, and decellularization of animal vessels.

Different biodegradable polymers have been extensively studied in the field of vascular tissue engineering with good results regarding their chemical-physical, mechanical, and biological properties. The polymers employed include natural polymers such as hyaluronic acid, gelatin, and collagen, as well as synthetic polymers such as poly(caprolactone) (PCL), poly(glycerol sebacate) (PGS), polylactic acid (PLLA), and poly(lactic-co-glycolic acid) (PLGA). Recently, in the international scientific scenario, biomaterials are considered not only as a simple scaffold in which cells will grow to form the regenerated tissue but also as drug delivery systems for bioactive molecules. This is made possible by the fact that scaffolds can be functionalized with various compounds responsible for the prevention and modulation of inflammatory phenomena that are the basis of the failure of bioprosthesis implantation, cell recruitment, and induction of their proliferation and/or trans-differentiation.

Currently, the technical problems related to the implantation of prostheses in vascular surgery are several:
Inability to have small caliber prostheses to be used in the surgical treatment of blood vessels in the upper and lower limbs;
Inability to have small caliber prostheses with greater long-term patency (absence of early post-implant thrombosis);
Inability to have biodegradable prostheses that avoid subjecting the patient to repeated surgeries in the event of treatment failure with the need for replacement of prostheses currently available on the market;
Inability to have bioactive prostheses, i.e., functionalized with biomolecules with anti-inflammatory activity and trans-differentiation factors capable of modulating the inflammatory process, a consequence of the surgical intervention, and inducing endothelialization of the employed construct.

### SUMMARY OF THE INVENTION

Engineered biodegradable vascular bioprostheses according to the invention are constituted by a polymeric construct containing a mixture of at least two polymers, poly(caprolactone) (PCL) and poly(glycerol sebacate) (PGS), and in which bioactive molecules (biomolecules) are present, chosen from antioxidants, which perform an activity of inflammation modulation, and from proteins, which perform an activity of cell recruitment and trans-differentiation.

The related method for obtaining such a polymeric construct comprises the preparation of a polymeric solution by solubilizing at least two polymers used in mixtures of suitable organic solvents, electrospinning the polymeric solution, and bioengineering.

### BRIEF DESCRIPTION OF THE DRAWINGS

The invention will be described below in at least one preferred embodiment for illustrative purposes only and not limiting the scope of the present invention, with the aid of the attached figures, in which:

FIG. 1 shows a schematic view of the cross-sectional structure of one embodiment of the bioprosthesis according to the invention; and

FIG. 2 illustrates a histogram of the results of hemocompatibility tests.

### DEFINITIONS

In the present document, the terms “about” or “around” as used herein when referring to a measurable value such as a quantity, a time duration, and the like, are intended to encompass variations from ±10% to ±0.1%, preferably ±10%, ±5%, ±1%, or ±0.1% from the specified value, where such variations are appropriate for performing the described methods.

In the present document, the quantity of each ingredient expressed as a range of values is to be understood as including the values at the boundaries of the range itself. Therefore, if an ingredient is between 10% and 20%, the endpoints of the range are included within the range itself.

### DETAILED DESCRIPTION OF EMBODIMENTS OF THE INVENTION

The present invention relates to engineered biodegradable vascular bioprostheses and the production process thereof.

The implantation of commercially available prostheses in patients affected by peripheral obstructive vascular diseases typically generates an inflammatory-scarring process that leads to the transformation of the prosthetic implant into a rigid and non-endothelialized structure, thereby promoting thrombotic processes, especially for small caliber prostheses, and, in the case of pathogen implantation during bacteremia or sepsis, the inability to eradicate the infection without explant. To reduce and modulate the inflammatory and thrombotic responses, targeted therapeutic protocols are employed. Furthermore, these commercial prostheses are not biodegradable, not bioactive, and are not ideally suited for the replacement of small caliber vessels. For these reasons, the use of biodegradable and engineered prostheses (bioprostheses) could ensure greater durability and patency of the implant over time. The success of the vascular bioprosthesis implant is closely dependent on its biodegradability, i.e., the time interval during which it will perform its task before being replaced by a neovessel, without undergoing thrombosis and calcification.

For these reasons, various methods have been defined to date, although with limited success, for the production of small caliber biodegradable vascular prostheses. Among the synthesis techniques reported in the limited literature on the subject, electrospinning with all its variants, 3D printing, and decellularization of animal vessels are mentioned.

Different biodegradable polymers have been extensively studied in the field of vascular tissue engineering with good results regarding their chemical-physical, mechanical, and biological properties. The polymers employed include natural polymers such as hyaluronic acid, gelatin, and collagen, as well as synthetic polymers such as poly(caprolactone) (PCL), poly(glycerol sebacate) (PGS), polylactic acid (PLLA), and poly-lactic acid-co-glycolide (PLGA). Recently, in the international scientific scenario, biomaterials are no longer considered merely as scaffolds in which cells must grow to form regenerated tissue, but also as drug delivery systems for bioactive molecules. This is made possible by the fact that scaffolds can be functionalized with various compounds responsible for the prevention and modulation of inflammatory phenomena that underlie the failure of bioprosthesis implants, the recruitment of cells, and the induction of their proliferation and/or trans-differentiation.

Currently, the technical problems related to the implantation of prostheses in vascular surgery are various:
inability to have small caliber prostheses for use in the surgical treatment of upper and lower limb vessels;
inability to have small caliber prostheses with greater long-term patency (absence of early thrombosis post-implant);
inability to have biodegradable prostheses that avoid subjecting the patient to repeated surgeries in the event of treatment failure, requiring replacement of the prostheses currently available on the market;
inability to have bioactive prostheses, i.e., functionalized with biomolecules with anti-inflammatory activity and trans-differentiation factors capable of modulating the inflammatory process, a consequence of the surgical procedure, and inducing endothelialization of the implanted construct.

Biodegradable engineered vascular bioprostheses with an internal diameter of less than 6 mm have now been obtained, produced by combining poly(caprolactone) (PCL) with poly(glycerol sebacate) (PGS) and optionally a third polymer, i.e., a polycaprolactone containing free carboxyl groups (PCL-s), and functionalized with bioactive molecules such as anti-inflammatory agents, cellular recruitment proteins, and proteins with cellular trans-differentiation activity that will respectively modulate the post-implant inflammatory process, recruit appropriate blood cells, and induce their trans-differentiation to favor endothelialization of the implanted prosthesis.

PCL is one of the most widely used polymers for the production of vascular prostheses, being hydrophobic and biocompatible. PGS is an innovative polymer, hydrophilic and also biocompatible.

PCL-s is a polycaprolactone-based polymer with an organic backbone modified by the grafting of a plurality of free carboxyl functionalities.

The combination of these polymers allows the advantages of each of the two or possibly three to be combined, resulting in bioresorbable vascular prostheses that can be slowly degraded (reabsorbed) over time and replaced by a neostructured vessel.

The ability to modulate the inflammatory process through the release of a bioactive molecule directly from a biodegradable prosthesis represents one of the main innovative and original features of this invention.

Another strength of the described invention lies in the functionalization of bioprostheses with proteins capable of inducing the differentiation of monocytes into endothelial cells, promoting endothelialization of the implanted prosthesis. The bioactive prosthesis, in the early stages of implantation, will be able to counteract the inflammatory processes induced by the implant itself. This will be possible due to the release of antioxidant and anti-inflammatory molecules with which the bioprostheses have been functionalized during the synthesis process. The endothelialization of the bioprosthesis, on the other hand, will be promoted by the presence of proteins covalently bound to the inner surface of the construct, which will direct the monocytes towards an endothelial cell lineage.

Furthermore, the originality of the presented invention also consists of the possibility of having available small caliber bioprostheses, preferably with an internal diameter of less than 6 mm, which are currently unavailable in vascular surgery.

These functionalized bioprostheses can represent innovative medical devices that address the inadequacy of synthetic prostheses currently used in vascular surgery.

The object of the present invention is biodegradable, biocompatible, and hemocompatible vascular bioprostheses. The vascular bioprostheses of the invention are multifunctional, i.e., capable of performing multiple biological activities simultaneously, and are used both in vascular surgery and regenerative medicine. These features allow overcoming the limitations of currently available prostheses and promote a more efficient and complete regeneration of pathological blood vessels.

The bioprosthesis according to the present invention comprises or is constituted by:
a polymeric construct;
at least one impermeable coating;
at least one anti-inflammatory agent, preferably antioxidant;
at least one cell recruiting agent;
at least one trans-differentiation cellular agent.

Preferably, said bioprosthesis has a cylindrical geometry.

Preferably, said bioprosthesis has an internal diameter ranging from 1.0-6.0 mm, preferably from 1.0-5.0 mm, more preferably from 2.0-4.0 mm.

In a preferred embodiment, said bioprosthesis has an internal diameter of about 2 mm.

Preferably, the walls of said bioprosthesis have a thickness ranging from 0.1 to 0.6 mm, preferably from 0.2 to 0.4 mm, more preferably from 0.25 to 0.35 mm.

Preferably, said bioprosthesis has a length ranging from 1.0 to 10.0 cm, preferably from 1.5 to 9.0 cm, more preferably from 2.0 to 8.0 cm.

The construct according to the invention represents the central core of the vascular bioprosthesis and is made of biodegradable and biocompatible polymers. As such, this polymeric core is biologically inactive and constitutes a structural scaffold to be functionalized with appropriate bioactive agents such as anti-inflammatory agents, antioxidants, and protein factors.

In some embodiments, said construct comprises or is constituted by a combination of:
polycaprolactone (PCL); and
poly(glycerol sebacate) (PGS).

Preferably, the volume or mass ratio between polycaprolactone (PCL) and poly(glycerol sebacate) (PGS) is preferably between 4 and 0.5, more preferably between 3 and 1, most preferably about 1.

In a preferred embodiment, said construct comprises the polymers Polycaprolactone (PCL) and Poly(glycerol sebacate) (PGS) in a volumetric or mass ratio of 1:1. Said PCL and PGS are preferably derived from polymer solutions prepared separately with a 20% m/v polymer concentration. Polymer concentrations of 10% m/v or 15% m/v may also be provided.

The construct according to the present embodiments further comprises at least one linker. The polymeric scaffold of the construct, in addition to not having any intrinsic biological activity, is devoid of chemically reactive surface functional groups suitable for post-functionalization, e.g., direct conjugation of proteins. Therefore, as a post-manufacturing strategy, in the present embodiments of the bioprosthesis, it may be advantageous to use at least one linker. Said linker provides reactive functional groups to immobilize both at least one trans-differentiation protein factor and at least one cell recruiting agent on the construct itself, particularly on the inner wall of said construct.

Said linker comprises at least one chemical group capable of forming a covalent bond with the protein to be bound.

These linkers form a bridge between the polymers of the construct on which hydroxyl and/or carboxyl groups (derived from the linker itself) have been grafted, and the protein that will be covalently bound to the linker and therefore to the polymer.

The linker may also not be directly contained in the bioprosthesis, but grafted onto a possible coating of the bioprosthesis itself, such as gelatin, for covalent binding with the proteins.

Preferably, said linker contains hydroxyl and/or carboxyl groups, such as lysine.

Preferably, said linker is a poly L-lysine (PLL). Said PLL is capable of strongly binding to chemically non-functionalizable polymeric surfaces, such as those comprising or constituted by mixtures of PCL and PGS.

In some preferred embodiments, said polymeric construct comprises or consists of a combination of three polymers:
Polycaprolactone (PCL);
Poly(glycerol sebacate) (PGS); and
Polycaprolactone enriched with carboxyl functionalities (PCL-s).

According to the present invention, the volumetric or mass ratio between Poly(caprolactone) (PCL) and Poly(glycerol sebacate) (PGS) is preferably between 4 and 0.5, preferably between 3 and 1, more preferably around 1.

In a preferred embodiment, said construct comprises:
PGS in an amount between 40-60% wt. (“% wt.” indicates the percentage ratio between the mass of the ingredient and the total mass of the construct), preferably between 45-55% wt.;
PCL in an amount between 30-50% wt., preferably between 35-45% wt.; and
PCL-s in an amount between 3-18% wt., preferably between 7-14% wt.

In a preferred embodiment, said construct comprises the polymers PCL, PGS, and PCL-s in a mass ratio of approximately

PGS:PCL:PCL-s=5:4:1

Said construct is preferably obtained from solutions of PGS, PCL, and PCL-s, preferably where the volumetric ratio between the volume of the 20% m/v PGS solution and the volume of the 20% m/v PCL and PCL-s solution is 1:1.

In a preferred embodiment, the volumetric ratio between the volume of a 20% m/v PGS solution and the volume of a 20% m/v PCL and PCL-s solution, where said PCL and PCL-s solution contains 80% m/v PCL and 20% m/v PCL-s, is 1:1.

In another preferred embodiment, the volumetric ratio between the volume of a 15% m/v PGS solution and the volume of a 15% m/v PCL and PCL-s solution, where said PCL and PCL-s solution contains 80% m/v PCL and 20% m/v PCL-s, is 1:1.

In another preferred embodiment, the volumetric ratio between the volume of a 10% m/v PGS solution and the volume of a 10% m/v PCL and PCL-s solution, where said PCL and PCL-s solution contains 80% m/v PCL and 20% m/v PCL-s, is 1:1.

To overcome the problem of the chemical inertness of the core surface, the inventors have designed and produced a construct with protruding carboxyl branches (derived from PCL-s) capable of covalently anchoring the proteins of interest. Advantageously, the use of PCL-s has allowed the creation of a central polymeric scaffold with structural properties similar to or at least analogous to the PCL and PGS mixture, lacking external linkers (e.g., polylysine) but already possessing available, exposed, and reactive chemical functionalities for the synthesis of molecular adducts. These carboxyl functionalities, particularly those located on the inner wall of the construct, therefore behave as reactive terminals for the immobilization of various compounds, preferably proteins.

The bioprosthesis according to the invention comprises at least one anti-inflammatory agent.

The implantation of a vascular prosthesis inevitably induces an acute inflammatory response, characterized by oxidative stress and the production of reactive oxygen species, which can compromise the integration of the device and negatively interfere with the resorption of the bioprosthesis.

This response can be modulated by various molecules such as antioxidants that exert an anti-inflammatory activity.

In a preferred embodiment of the present invention, said at least one anti-inflammatory agent, preferably antioxidant, is incorporated/contained/embedded/dispersed within the polymeric matrix of the construct. According to this embodiment, it is possible to modulate the inflammatory process through the controlled release of the bioactive antioxidant molecule over time. This release occurs concomitantly with the biodegradation of the vascular prosthesis of the invention.

The bioactive prosthesis, especially in the early stages of implantation, will thus be able to counteract the inflammatory processes induced by the implant itself.

Said anti-inflammatory agent is preferably a bioactive antioxidant molecule. The antioxidants that exert and modulate an anti-inflammatory activity can preferably be chosen from polyphenols.

These represent a very broad class of chemically different compounds that exert antioxidant and anti-inflammatory activity.

The at least one antioxidant agent may be selected by way of example and not limitation from:
flavones, preferably apigenin;
flavonoids, preferably quercetin;
phenols, preferably resveratrol and tyrosol.

In a preferred embodiment, the at least one anti-inflammatory and antioxidant agent is quercetin.

The at least one anti-inflammatory agent, during the preparation of the polymeric construct, is added to the polymeric mixture.

Said at least one anti-inflammatory agent added to the polymeric electrospinning mixture must be present in a concentration such that, once released from the scaffold, it is in biologically active quantities.

Said at least one anti-inflammatory agent is added to the polymeric electrospinning mixture in an amount preferably between 0.01-0.05% m/v, preferably between 0.02-0.05% m/v, preferably between 0.03-0.05% m/v, preferably between 0.04-0.05% m/v, more preferably about 0.05% m/v, with the solvent of said electrospinning polymeric mixture preferably consisting of chloroform and ethanol, preferably in a volumetric ratio of 9:1.

Said anti-inflammatory agent is preferably dispersed in the polymeric matrix of the construct.

Preferably, said anti-inflammatory agent is dispersed in the matrix of the construct in an amount between 0.01-0.125% wt. (i.e., the percentage ratio of the mass of the anti-inflammatory agent to the mass of the construct alone).

The bioprosthesis according to the invention comprises a impermeable agent.

The polymeric construct is intrinsically porous, a characteristic that results in high permeability to fluids, particularly aqueous ones (e.g., blood). Since the bioprostheses of the present invention are intended for use in pathological blood vessels, excessive porosity may cause blood leakage through the wall (blood leakage), leading to a risk of hemorrhage and immediate complications. Furthermore, excessive porosity may hinder the rapid formation of an internal endothelial coating and promote the uncontrolled penetration of cells into the structure.

The inventors have solved the above technical problems by superficially coating the polymeric construct with a water-repellent substance, preferably applied to the external surface of said construct.

Said impermeable agent may be selected by way of example and not limitation from the group comprising gelatin, collagen, fibronectin, vitronectin.

In a preferred embodiment, said impermeable agent is gelatin, i.e., denatured collagen, preferably of bovine origin.

The bioprosthesis of the invention comprises a trans-differentiation cellular agent.

In addition to modulating the inflammatory process, it is important that the implanted prosthesis be able to induce endothelialization of its internal walls to promote regeneration of the blood vessel. This cellular process can be favored by the presence of molecules capable of inducing the differentiation of circulating blood cells into endothelial cells that will line the surface of the scaffold.

The at least one trans-differentiation agent included in the bioprosthesis of the invention is thus capable of directing endothelial progenitor cells to become vessel cells, i.e., induce a phenotypic conversion into functional endothelial cells.

In a preferred embodiment, said trans-differentiation agent is capable of converting blood monocytes into endothelial cells. The phenotypic-functional instruction imparted to the monocyte cells will allow them to differentiate into endothelial cells and line the inner surface of the bioprosthesis, promoting endothelial regeneration of the replaced vessel.

Said at least one trans-differentiation agent is preferably disposed on the inner surface of the bioprosthesis. Once the bioprosthesis is applied to the damaged vascular channel or tube, said at least one trans-differentiation agent distributed along the lumen (inner wall) of the vascular prosthesis will be immersed in the bloodstream and may interact with the blood monocyte cells.

Said at least one trans-differentiation agent may be immobilized on the bioprosthesis or introduced and dispersed among the polymeric fibers for slow and controlled release.

Said at least one trans-differentiation agent is preferably covalently bound to the polymeric matrix of the construct, preferably to the inner surface of said construct. Advantageously, the establishment of a covalent chemical bond to the polymeric construct prevents the blood flow within the vessel from washing away said trans-differentiation agent. The covalent immobilization allows this differentiation signal to maintain its effectiveness over time, acting locally without being subject to degradation or systemic diffusion.

In a particularly preferred embodiment, said at least one trans-differentiation agent is chemically bound to the carboxyl groups of PCL-s. Said trans-differentiation agent, covalently bound to the inner surface of the construct, will be present in sufficient quantity, preferably in excess, to allow all endothelial precursors (monocytes), once they arrive on the prosthesis, to activate the biochemical processes that lead to differentiation into endothelial cells.

Said at least one trans-differentiation cellular agent is preferably selected from:
Pleiotrophin (PTN);
Vascular Endothelial Growth Factor (VEGF-A165);
Basic Fibroblast Growth Factor (bFGF/FGF-2) in combination with VEGF;
Chemokine CXCL12 (SDF-1α);
Angiopoietin-1 (ANGPT1) in combination with at least one of VEGF and FGF;
and their combinations.

VEGF-A165 is the main molecule for the endothelialization process and can recruit circulating monocytes onto functionalized surfaces, even in the presence of shear stress. When present with other angiogenic factors, VEGF-A165 promotes the acquisition of typical endothelial cell markers.

bFGF/FGF-2 supports the proliferation/survival of cells and, when combined with VEGF, supports the maturation of endothelial-like cells.

CXCL12 is a powerful molecule for recruitment and pro-vascular maturation. As a local signal, it supports pro-endothelial activation. CXCL12, when combined with heparin in the bioprosthesis of the invention, improves the recruitment of endothelial progenitors and the integration of vascular tissue.

ANGPT1 in combination with at least one of VEGF/FGF helps to maintain a typical endothelial cell phenotype and cell junction characteristics (Tie2).

In a preferred embodiment, said trans-differentiation agent is pleiotrophin (PTN).

Said bioprosthesis according to the invention comprises at least one cellular recruitment agent.

To enable the proper functioning of said at least one trans-differentiation cellular agent, the inventors have recognized the need to functionalize the bioprosthesis of the invention with at least one chemotactic agent capable of attracting specific blood cells and making them available to the trans-differentiation agent. Specifically, such recruitment agents attract specific circulating cell populations, such as blood cells, preferably monocytes, preventing the blood flow from dispersing them away from the implanted device.

Advantageously, the anti-inflammatory action promoted by said at least one anti-inflammatory agent simultaneously favors the recruitment activity of said cellular recruitment agent. Indeed, the timed release of the antioxidant agent ensures that cellular recruitment occurs in a more stable and less hostile tissue context, maximizing the efficiency of cellular colonization of the prosthetic surface.

Said cellular recruitment agents (chemotactic agents) are preferably immobilized on the polymeric construct, more preferably on the inner surface (inner wall or lumen) of said polymeric construct.

In some preferred embodiments, said cellular recruitment agents are immobilized by forming a covalent chemical bond.

In some particularly preferred embodiments, said cellular recruitment agents are covalently bound to the carboxyl functionalities of PCL-s in the polymeric construct.

The amount of the chemotactic factor for endothelial progenitors is defined such that it can recruit a sufficient number of endothelial progenitors (monocytes) so that vascular regeneration can occur.

Said at least one cell recruiting agent may be, by way of example, a peptide or a chemokine.

Preferably, said at least one cell recruiting agent is chosen from the group comprising:
MCP1 (Monocyte Chemoattractant Protein-1);
CCL7 (MCP-3);
CCL8 (MCP-2);
CCL13 (MCP-4);
CX3CL1 (Fractalkine);
and their combinations.

CCL7 is recognized by the receptors: CCR2, CCR1/CCR3, and has a broader action spectrum than MCP-1, advantageously useful for maximizing the engagement of “classic” monocytes.

CCL8 is recognized by the receptors: CCR2, CCR1/CCR5, and is combinable at low doses with MPC-1/MCP-3.

CCL13 is recognized by the receptors: CCR2, CCR3/CCR1, and has a similar behavior to MCP1 but is advantageously useful for avoiding tachyphylaxis using low-dose mixtures.

CX3CL1 is recognized by the receptor CX3CR1, is a chemokine both soluble and adhesive (membrane form), and is optimal for the capture of monocytes and their chemotaxis to achieve their local accumulation.

In a particularly preferred embodiment, said at least one cell recruiting agent is MCP1.

For all the reasons stated above, the functionalization of biomaterials with both antioxidant and/or anti-inflammatory molecules and endothelializing molecules (for example, capable of differentiating circulating monocytes into endothelial cells) and with cellular recruitment agents represents a highly promising solution to the problems that current prostheses face in vascular surgery.

The inventors have deliberately selected a combination of three bioactive components to functionalize the vascular prosthesis of the invention, i.e., cellular recruitment agent, trans-differentiation agent, and anti-inflammatory agent, in order to biomimetically reproduce the physiological stages of healing and vascular integration following implantation. Each component is selectively chosen to intervene at a precise moment of the biological response and with a complementary action to the others, in an orchestrated process that allows overcoming the limitations of traditional vascular devices.

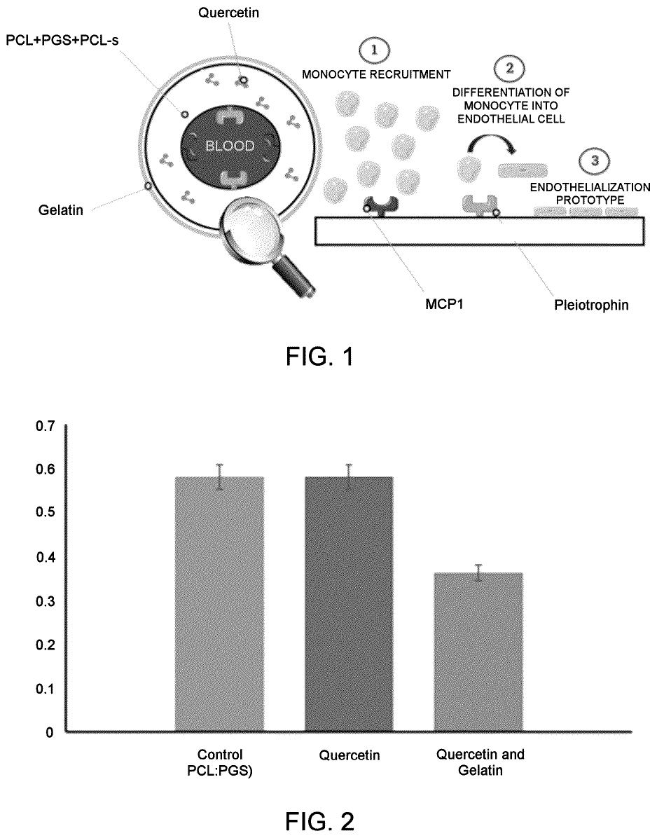

In a preferred embodiment of the bioprosthesis of the invention illustrated in FIG. 1, said bioprosthesis comprises or consists of:
a polymeric construct comprising a mixture of PCL, PGS, and PCL-s;
gelatin;
quercetin;
MCP1;
Pleiotrophin;
in which the gelatin coats the external wall of said bioprosthesis, quercetin is dispersed in the polymeric matrix of said construct, MCP1 and pleiotrophin are covalently bound to the inner surface of said bioprosthesis.

Advantageously, the bioprosthesis according to the invention has been shown to be perfectly biocompatible. As illustrated in FIG. 2, all the samples analyzed showed a hemolysis rate of less than 5%, the reference threshold value defined by international standards.

A second object of the invention is represented by the process for obtaining the engineered biodegradable vascular bioprostheses in accordance with the invention.

In detail, the process for obtaining the engineered vascular bioprostheses comprises the following stages:
preparation of a polymeric solution by solubilizing polycaprolactone (PCL) and poly(glycerol sebacate) (PGS) in mixtures of suitable organic solvents, and optionally polycaprolactone enriched with carboxyl functionalities (PCL-s);
electrospinning of the obtained polymeric solution;
bioengineering of the vascular bioprostheses;
the preparation of the polymeric solution is preferably obtained by separately preparing:
the PGS solution; and
the PCL solution or the PCL and PCL-s solution;
and then mixing them, preferably in a volumetric ratio of 1:1;
in which, preferably, the preparation of the polymeric solution of poly(glycerol sebacate) (PGS) is carried out at a concentration between 10% and 30% (m/v) in a solution of suitable organic solvents; and
the preparation of the polymeric solution of polycaprolactone (PCL) is carried out at a concentration between 10% and 30% (m/v) in a solution of suitable organic solvents, or the preparation of the PCL and PCL-s solution is carried out at a concentration between 10% and 30% (m/v) in a solution of suitable organic solvents, in which the mass ratio between PCL and PCL-s is approximately

PCL:PCL-s=80:20
said two solutions obtained are stirred, preferably by a magnetic stirrer, and then mixed.

The solution of suitable organic solvents for the preparation of the polymeric solution of poly(caprolactone) (PCL) and/or the solution of suitable organic solvents for the preparation of the polymeric solution of poly(glycerol sebacate) (PGS) and/or the polymeric solution of poly(caprolactone) enriched with carboxyl functionalities (PCL-s) preferably contains or is composed of chloroform and ethanol in a volumetric ratio between 8/1 and 10/1, the ethanol used may contain dissolved bioactive molecules (biomolecules) chosen from the antioxidants that exert an anti-inflammatory activity in one or both solutions.

The concentration in ethanol of bioactive molecules (biomolecules) chosen from antioxidants that exert an anti-inflammatory activity, in one or both solutions, if present, is preferably between 4 and 7 mg/mL, with this concentration in the two solutions possibly differing.

The bioactive molecules (biomolecules) are chosen from antioxidants that exert an anti-inflammatory activity, preferably from polyphenols, more preferably from flavones, in particular apigenin, flavonoids, in particular quercetin, phenols, in particular resveratrol and tyrosol.

Quercetin is the preferred and recommended polyphenol.

The electrospinning must be carried out in such a way as to obtain a polymeric construct that possesses suitable chemical-physical, mechanical, biological, and suturability characteristics.

Said electrospinning stage takes place under the following preferred conditions:
flow of the electrospun solution between 0.60 and 2.20 mL/h;
voltage between 14 and 20 k Volt;
external diameter of the collector between 1 and 6 mm;
needle-collector distance between 15 and 18 cm;
volume of the electrospun solution between 1.5 and 2.0 mL;
collector rotation speed between 400 and 800 rpm;
collector translation speed between 350 and 650 mm/min.

By varying the parameters of the electrospinning process within the ranges indicated above, bioprostheses varying between 1 and 6 mm in internal diameter can be obtained, with the best possible performances in terms of chemical-physical, mechanical, suturability, and biological activity properties.

The bioengineering of the obtained bioprostheses can also take place using a linker that will allow covalent bonding between the functional groups of the polymer and the trans-differentiation protein factor.

The bioengineering can be either simultaneous to the electrospinning stage, for example, by adding the antioxidant to the two polymers or adding nanoparticles that encapsulate either polyphenols or trans-differentiation proteins to the polymeric solution, or subsequent to the electrospinning stage.

The bioengineering of the obtained bioprostheses, carried out downstream of the electrospinning stage of the polymeric solution, preferably takes place by coating the obtained bioprosthesis with a compound, such as gelatin, and by grafting the aforementioned linker containing hydroxyl and/or carboxyl groups to coat the inner surface of the bioprosthesis.

The immobilization (chemical anchoring) of said at least one cell recruiting agent and/or said at least one trans-differentiation cellular agent to the polymeric construct can be carried out according to techniques well known to a person skilled in the art.

In a preferred embodiment, said at least one cell recruiting agent and/or said at least one trans-differentiation cellular agent are covalently bound to said polymeric construct through the carboxyl groups of PCL-s.

Said covalent bond is preferably formed through a chemical reaction with EDC (1-ethyl-3-(3-dimethylaminopropyl) carbodiimide) and NHS (N-hydroxysuccinimide). The experimental procedure is within the reach of any skilled person in the field.

The procedure for coating said polymeric construct of the bioprosthesis of the invention with a impermeable agent can also be carried out using experimental procedures known to an average technician in the field.

A further object of the invention is represented by the use of the engineered vascular bioprostheses as described above as medical devices.

### EXAMPLES

### Study of Hemocompatibility of Vascular Prostheses

The inventors conducted a hemocompatibility study of the bioprosthesis of the invention to determine its suitability for application in the vascular system.

For the following study, three variants of bioprostheses (hereinafter referred to as scaffolds) of the invention were used:
“Control” bioprosthesis (C) consisting of the polymeric construct made from a mixture of PCL and PGS;
“Quercetin” bioprosthesis (Q) consisting of a polymeric construct in PCL and PGS, functionalized with quercetin;
“Quercetin and Gelatin” bioprosthesis (QG) consisting of a polymeric construct in PCL and PGS, functionalized with quercetin and coated with gelatin.

For haemocompatibility testing, blood was collected from healthy volunteers via venipuncture into vacutainer tubes containing ethylenediaminetetraacetate (EDTA) as an anticoagulant.

For the hemolysis test, each scaffold was cut (surface of 50 mm²) and rinsed three times with deionized water. Subsequently, the tests were performed by placing each scaffold in a 0.9% NaCl solution or in deionized water. To this solution, 0.1 mL of fresh human blood was then added. After 30 minutes, the erythrocytes ruptured their membranes, and hemoglobin was released into the solution.

Similarly, 0.1 mL of fresh blood was added to 5 mL of distilled water and to 0.9% NaCl, which were used as positive and negative controls, respectively. The positive and negative controls are necessary to exclude erythrocyte rupture phenomena not directly caused by the samples.

At the end of the incubation time (30 min), all the samples were centrifuged at 2000 rpm for 5 minutes. The supernatants were collected and transferred to cuvettes to measure the absorbance at 540 nm (Lambda 25 spectrophotometer, PerkinElmer, Milan, Italy), the maximum absorption of hemoglobin, one of the most abundant proteins expressed by erythrocytes.

The hemolysis rate (HR) was calculated as the ratio between the absorbance of the sample and the two controls, negative and positive.

As evident from FIG. 2, all the samples studied showed a low hemolysis percentage, i.e., a value lower than 5% (threshold value considered by international standards).

The invention claimed is:

1. A biodegradable engineered vascular bioprosthesis comprising:
a polymeric construct;
an impermeable coating;
an anti-inflammatory agent;
a cell recruiting agent; and
a trans-differentiation cellular agent.

2. The biodegradable engineered vascular bioprosthesis according to claim 1, wherein the bioprosthesis has a cylindrical shape with an internal diameter ranging from 1.0 to 6.0 mm, a wall having a thickness ranging from 0.1 to 0.6 mm, and a length ranging from 1.0 to 10.0 cm.

3. The biodegradable engineered vascular bioprosthesis according to claim 1, further comprising a linker for conjugation with the cell recruiting agent and/or the trans-differentiation cellular agent, wherein the polymeric construct comprises polycaprolactone (PCL) and poly(glycerol sebacate) (PGS).

4. The biodegradable engineered vascular bioprosthesis according to claim 3, wherein the linker contains hydroxyl and/or carboxyl groups.

5. The biodegradable engineered vascular bioprosthesis according to claim 1, wherein the polymeric construct comprises polycaprolactone (PCL), poly(glycerol sebacate) (PGS), and polycaprolactone enriched with carboxyl functional groups (PCL-s).

6. The biodegradable engineered vascular bioprosthesis according to claim 5, wherein:
the poly(glycerol sebacate) is present in an amount ranging from 40 to 60% wt. relative to a mass of the polymeric construct;
the polycaprolactone (PCL) is present in an amount ranging from 30 to 50% wt. relative to the mass of the polymeric construct; and
the polycaprolactone enriched with carboxyl functional groups (PCL-s) is present in an amount ranging from 3 to 18% wt. relative to the mass of the polymeric construct.

7. The biodegradable engineered vascular bioprosthesis according to claim 5, wherein the PGS, PCL, and PCL-s are present in a mass ratio of approximately
PGS:PCL:PCL-s=5:4: 1.

8. The biodegradable engineered vascular bioprosthesis according to claim 1, wherein the anti-inflammatory agent is an antioxidant dispersed in a polymeric matrix of the polymeric construct.

9. The biodegradable engineered vascular bioprosthesis according to claim 1, wherein the anti-inflammatory agent is selected from:
flavones;
flavonoids; and
phenols.

10. The biodegradable engineered vascular bioprosthesis according to claim 1, wherein at least one impermeable agent coats an outer surface of a wall of the polymeric construct.

11. The biodegradable engineered vascular bioprosthesis according to claim 10, wherein the impermeable agent is gelatin, collagen, fibronectin, or vitronectin.

12. The biodegradable engineered vascular bioprosthesis according to claim 1, wherein the trans-differentiation cellular agent is covalently bonded to an inner surface of a wall of the polymeric construct.

13. The biodegradable engineered vascular bioprosthesis according to claim 1, wherein the trans-differentiation cellular agent is selected from:
pleiotrophin (PTN);
vascular endothelial growth factor (VEGF-A165);
basic fibroblast growth factor (bFGF/FGF-2) in combination with VEGF;
chemokine CXCL12 (SDF-1α);
angiopoietin-1 (ANGPT1) in combination with at least one of VEGF and FGF;
and combinations thereof.

14. The biodegradable engineered vascular bioprosthesis according to claim 1, wherein the cell recruiting agent is immobilized on an inner surface of a wall of the construct.

15. The biodegradable engineered vascular bioprosthesis according to claim 1, wherein the cell recruiting agent is selected from:
MCP1 (Monocyte Chemoattractant Protein-1);
CCL7 (MCP-3);
CCL8 (MCP-2);
CCL13 (MCP-4);
CX3CL1 (Fractalkine);
and combinations thereof.

16. The biodegradable engineered vascular bioprosthesis according to claim 1, wherein:
the polymeric construct comprises PGS, PCL, and PCL-s;
the impermeable coating is gelatin;
the anti-inflammatory agent is quercetin;
the cell recruiting agent is MCP1; and
the trans-differentiation cellular agent is pleiotrophin.

17. A method of obtaining a biodegradable engineered vascular bioprosthesis according to claim 1, comprising:
preparing a polymeric solution by solubilizing polycaprolactone (PCL), poly(glycerol sebacate) (PGS), and optionally polycaprolactone enriched with carboxyl functional groups (PCL-s) in organic solvent mixtures;
electrospinning the obtained polymeric solution to obtain polymeric constructs; and
bioengineering the polymeric constructs to obtain the vascular bioprosthesis.

18. The method according to claim 17, wherein the polymeric solution is obtained by first shaking and then mixing, in a 1:1 volumetric ratio, a solution of PGS having a concentration ranging from 10 to 30% m/v in an organic solvent solution and a solution of PCL having a concentration ranging from 10 to 30% m/v in an organic solvent solution.

19. The method according to claim 17, wherein the polymeric solution is obtained by first shaking and then mixing, in a 1:1 volumetric ratio, a solution of PGS having a concentration ranging from 10 to 30% m/v in an organic solvent solution and a solution of PCL and PCL-s having a concentration ranging from 10 to 30% m/v in an organic solvent solution, wherein a mass ratio between PCL and PCL-s is 80:20.

20. The method according to claim 16, wherein the electrospinning is carried out under the following conditions:
a flow rate of the electrospun polymeric solution ranging from 0.60 to 2.20 mL/h;
a voltage ranging from 14 to 20 kVolt;
a collector having an outer diameter ranging from 1 to 6 mm;
the collector having a needle distance ranging from 15 to 18 cm;
a volume of the electrospun polymeric solution ranging from 1.5 to 2.0 mL;
a rotation speed of the collector ranging from 400 to 800 rpm; and
a translation speed of the collector ranging from 350 to 650 mm/min.
